(19) United States
(12) Patent Application Publication
(10) Pub. No.: US 2024/0264140 A1
(43) Pub. Date: Aug. 8, 2024

(54) MANUFACTURING METHOD OF RESIN CONCRETE DEGRADATION MODEL, PREDICTION METHOD OF RESIN CONCRETE DEGRADATION, AND RESIN CONCRETE DEGRADATION MODEL

(71) Applicant: NIPPON TELEGRAPH AND TELEPHONE CORPORATION, Tokyo (JP)

(72) Inventors: Hisatoshi KASAHARA, Tokyo (JP); Yosuke TAKEUCHI, Tokyo (JP); Yosuke OKAMURA, Tokyo (JP); Junichiro TAMAMATSU, Tokyo (JP)

(73) Assignee: NIPPON TELEGRAPH AND TELEPHONE CORPORATION, Tokyo (JP)

(21) Appl. No.: 18/565,039

(22) PCT Filed: May 31, 2021
(86) PCT No.: PCT/JP2021/020717

§ 371 Date: Nov. 28, 2023

(51) Int. Cl. G01N 33/38; G01N 3/20

(52) U.S. Cl. CPC G01N 33/383; G01N 3/20

(57) ABSTRACT

A method of producing a resin concrete deterioration model, a method of predicting deterioration of resin concrete, and a resin concrete deterioration model are provided. The method of producing a resin concrete deterioration model adds water to a precursor of resin concrete containing a thermosetting resin, calcium carbonate, and aggregate and kneads the mixture. The resin concrete deterioration model 1 includes a thermosetting resin, calcium carbonate, an aggregate, and water.

### TECHNICAL FIELD

The present invention relates to a method of producing a resin concrete deterioration model, a method of predicting deterioration of resin concrete, and a resin concrete deterioration model.

### BACKGROUND ART

Resin concrete is concrete made by firmly hardening aggregate, calcium carbonate, and the like using a thermosetting resin (resin) as a binder. As a binder resin, an unsaturated polyester resin which is inexpensive and has a curing time that can be freely adjusted is often used. Since a resin is used as a binder, the resin concrete is excellent in strength, moldability, and the like as compared to conventional concrete. Therefore, resin concrete is widely used as a material for forming a structure such as a manhole, a sewer pipe, and information system boxes. For example, 100,000 or more communication manholes formed of the resin concrete exist in Japan.

Resin concrete and characteristics thereof are described in NPL 1. NPL 1 describes that resin concrete is concrete made by firmly hardening crushed stone, sand, and calcium carbonate using a thermosetting resin (resin) as a binder without using cement and water. In addition, it is described that as a binder resin of the resin concrete, an unsaturated polyester resin having a curing time that can be freely adjusted is usually used, and an epoxy resin or the like is also used depending on the application. Further, it is described that the resin concrete is quickly cured and can obtain a predetermined strength in a short period of time, has a high strength and a high adhesiveness, and is a dense material and thus is excellent in water tightness and insulation, and is excellent in acid resistance. It is also described that the resin concrete has a strength of about 3 to 5 times higher than that of general cement concrete because a thermosetting resin is used as a binder.

Results of examination regarding deterioration of resin concrete are described in NPL 2. NPL 2 describes that it is not necessary to consider corrosion of a reinforcement, and it is conventionally assumed that when an unsaturated polyester resin is used for resin concrete, the resin may be hydrolyzed to lower the strength. Further, as new examination results, in addition to such reduction in the strength due to hydrolysis, it is described that deterioration of the resin concrete is caused by a high likelihood of strength deterioration due to water content and a strong negative primary correlation between a water content and a bending strength.

### CITATION LIST

### Non Patent Literature

[NPL 1] Characteristics of resin concrete, Sunrec, Co., [retrieved on May 24, 2021], Internet <https://www.sunrec.co.jp/about_concrete/>
[NPL 2] Takashi Miwa, Kazue Ichino Takahashi, Hiroyuki Takahashi, and Takashi Sawada, Strength Reduction Mechanism and Strength Estimation of Unsaturated Polyester Resin Concrete Deteriorated in Outdoor Soil, Materials and Environments, Vol. 69, No. 6. pp. 161-167 (2020)

### SUMMARY OF INVENTION

### Technical Problem

When the strength of a resin concrete structure deteriorates over time (hereinafter simply referred to as deterioration over time), there is a concern that the proof stress or strength (hereinafter simply referred to as strength) is affected. For an existing structure, a sample of a resin concrete portion is collected from the structure, and a bending test or a compression test (hereinafter simply referred to as strength evaluation) is performed according to JIS A 1181:2005, for example, to ascertain the present strength. By performing such evaluation, the strength and a deterioration state of the resin concrete structure can be accurately ascertained. However, in order to perform accurate strength evaluation, it is necessary to collect a predetermined shape and a predetermined amount of samples from the structure. Therefore, there are cases where a structure is small and thus is not suitable for collection of samples and cases where collection of samples is not suitable from the viewpoint of safety or the like due to a situation in which collection of samples lowers the strength of a structure, and the like. In addition, it is desirable to design a structure to be constructed using resin concrete assuming strength reduction in advance, and it is sometimes desired to ascertain strength after deterioration over time in advance.

In this manner, even when a sample cannot be collected from an existing structure or collection is not suitable, if characteristics of deterioration of the resin concrete over time can be ascertained by other means, the characteristics can be utilized for evaluation of safety of the existing structure. Further, if characteristics of deterioration of the resin concrete over time can be ascertained in advance, a structure to be constructed can be designed assuming strength reduction in advance.

However, a method of producing resin concrete deteriorated over time or a model for substituting resin concrete deteriorated over time has not been established. Therefore, it is desired to provide a method of ascertaining a mechanical strength of resin concrete after deterioration over time, that is, a method of predicting deterioration of the resin concrete, a resin concrete deterioration model (sample), and a method of producing the resin concrete deterioration model.

The present invention has been devised in view of such an actual situation, and an object of the present invention is to provide a method of producing a resin concrete deterioration model, a method of predicting deterioration of resin concrete, and a resin concrete deterioration model.

### Solution to Problem

A method of producing a resin concrete deterioration model according to the present invention for achieving the foregoing object includes adding water to a precursor of resin concrete containing a thermosetting resin, calcium carbonate, and aggregate and kneading the mixture.

The method of producing a resin concrete deterioration model according to the present invention further may further include:
a mixing step of mixing the thermosetting resin, the calcium carbonate, and the aggregate to prepare the precursor; and
a kneading step of adding water to the precursor and kneading the mixture.

The method of producing a resin concrete deterioration model according to the present invention further may further include:
increase the addition amount of water when a deterioration model simulating a case of an older material age is produced.

A method of predicting deterioration of resin concrete according to the present invention for achieving the foregoing object includes:
a model producing process of producing a resin concrete deterioration model containing a thermosetting resin, calcium carbonate, and aggregate;
a strength evaluation process of acquiring the strength of the resin concrete deterioration model;
a data accumulation process of producing two or more resin concrete deterioration models having different addition amounts of water in the model producing process and executing the strength evaluation process to acquire a value of the strength corresponding to the addition amount of water; and
a correlation formula acquisition process of acquiring a correlation formula of the strength with respect to the addition amount on the basis of acquired two or more strengths and addition amounts corresponding to the two or more strengths.

Furthermore, the method of predicting deterioration of resin concrete according to the present invention may produce the resin concrete deterioration model deteriorated to a target strength on the basis of the correlation formula.

Furthermore, the method of predicting deterioration of resin concrete according to the present invention may obtain a target addition amount of water corresponding to the target strength on the basis of the correlation formula, and add the target addition amount of water to a precursor of resin concrete containing the thermosetting resin, the calcium carbonate, and the aggregate to produce the resin concrete deterioration model.

Furthermore, in the method of predicting deterioration of resin concrete according to the present invention, the strength may be a bending strength.

A resin concrete deterioration model according to the present invention for achieving the foregoing object includes:
a thermosetting resin;
calcium carbonate;
an aggregate; and
water.

### Advantageous Effects of Invention

It is possible to provide a method of producing a resin concrete deterioration model, a method of predicting deterioration of resin concrete, and a resin concrete deterioration model.

### BRIEF DESCRIPTION OF DRAWINGS

FIG. 1 is a graph of strain-bending stress of resin concrete of each material age.

FIG. 2 is a diagram showing a shape of a resin concrete deterioration model.

FIG. 3 is a graph obtained by superimposing a graph of strain-bending stress of a sample with 2% added water on the graph of FIG. 1.

FIG. 4 is a graph showing a correlation between an amount of water added and a bending strength.

FIG. 5 is a flowchart in the case of producing a resin concrete deterioration model for predicting deterioration of resin concrete.

FIG. 6 is a diagram showing another shape of a resin concrete deterioration model.

### DESCRIPTION OF EMBODIMENTS

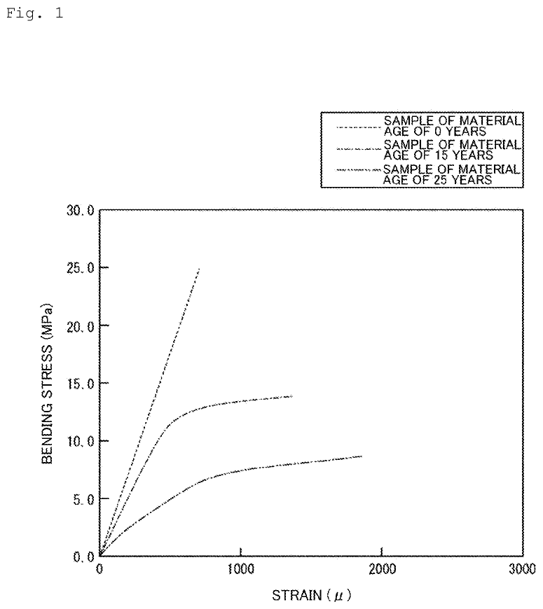

Prior to description of embodiments of the present invention, aging deterioration of resin concrete will be described. FIG. 1 shows results of bending strength tests of samples collected from resin concrete of each material age. FIG. 1 shows the relationship between a strain and a bending stress obtained on the basis of “bending strength test” described in section 8.3 of JIS A 1181:2005 as a graph. In FIG. 1, a sample collected from resin concrete after 0 years from production is described as “sample of material age of 0 years,” and samples collected from resin concrete after 15 years and 25 years are described as “sample of material age of 15 years” and “sample of material age of 25 years.” It can be ascertained from FIG. 1 that a sample of resin concrete of an old material age (hereinafter, it may be simply referred to as a sample) has a reduced strength, that is, has deteriorated over time as a result of the elapse of a predetermined number of years from production.

Each sample behaves differently when broken depending on the material age thereof. In FIG. 1, the sample of the material age of 0 years has broken after elastic deformation. On the other hand, the samples of the material age of 15 years and the material age of 25 years whose strengths have been lowered over time have a small breaking stress and a large strain at the time of breaking. That is, resin concrete which has a young material age and has not deteriorated over time exhibits an elastic fracture behavior. On the other hand, resin concrete which has an old material age and a reduced strength due to deterioration over time exhibits an elastoplastic fracture behavior.

If it is possible to provide a resin concrete deterioration model capable of reproducing the characteristic mechanical strength and fracture characteristics of the above-described resin concrete instead of the resin concrete deteriorated over time, and the resin concrete deterioration model can be utilized for evaluation of safety of existing structures (for example, manholes, sewer pipes, information system boxes, and the like) even when samples cannot be collected from the existing structures or collection is not suitable. Further, if it is possible to provide such a resin concrete deterioration model, characteristics of deterioration of resin concrete over time can be ascertained in advance and a structure to be constructed can be designed assuming strength deterioration in advance. That is, if it is possible to provide a resin concrete deterioration model that simulates or reproduces the mechanical strength of resin concrete depending on the material age and is an alternative to mechanical evaluation of resin concrete which has actually deteriorated over time, safety of existing structures can be easily evaluated, and a structure to be constructed can be designed assuming strength reduction.

Hereinafter, a method of producing a resin concrete deterioration model, a method of predicting deterioration of resin concrete, and a resin concrete deterioration model according to an embodiment of the present invention will be described with reference to the drawings.

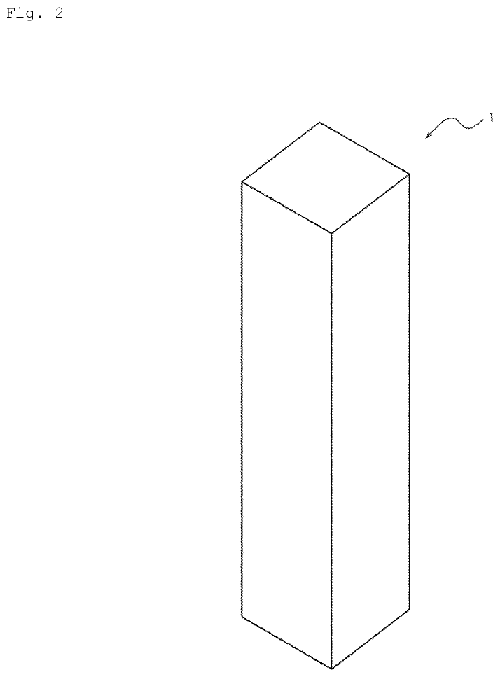

FIG. 2 shows a resin concrete deterioration model 1 (hereinafter referred to as deterioration model 1) according to the present embodiment. The deterioration model 1 is a model (for example, a test piece or a sample) simulating a mechanical strength of resin concrete of a predetermined material age. The deterioration model 1 contains at least a thermosetting resin, calcium carbonate, and an aggregate, and further contains water according to a material age for simulation. The deterioration model 1 is produced by adding water to a precursor of resin concrete containing a thermosetting resin, calcium carbonate, and an aggregate and kneading the same. A deterioration model 1 of a material age of 0 years does not contain water.

A method of producing the deteriorated model 1 (an example of a model producing process) will be described in detail. In producing the deterioration model 1, a mixing process for preparing a precursor of resin concrete is performed first. In the mixing process, the thermosetting resin, powder of calcium carbonate, and the aggregate are mixed to prepare the precursor. That is, the precursor is a mixture of the thermosetting resin, the calcium carbonate, and the aggregate. The aggregate is sand or gravel. One example of the thermosetting resin is an unsaturated polyester resin, a phenol resin, or an epoxy resin.

Table 1 below shows an example of a precursor formulation. The formulation of the precursor shown in FIG. 1 is the same as that of the resin concrete of each material age shown in FIG. 1.

TABLE 1

| | Mixing ratio |
|---|---|
| Name of material | (mass %) |
| Unsaturated | 10 |
| polyester resin | |
| Gravel | 50 |
| Sand | 20 |
| Calcium carbonate | 20 |

In the formulation shown in Table 1, a case in which the thermosetting resin is an unsaturated polyester resin is shown. The unsaturated polyester resin shown in Table 1 is a resin before heat curing and is a liquid resin. Although the items are not described separately in Table 1, a predetermined amount of curing agent is added to the unsaturated polyester resin shown in Table 1. The curing agent is, for example, a cross-linking monomer. Table 1 shows a case in which where gravel and sand are used as an aggregate.

Next, a kneading step of adding water to the precursor and kneading the mixture is performed. The deterioration model 1 having an arbitrary shape can be obtained by injecting the kneaded product obtained through the kneading process (hereinafter simply referred to as a kneaded product) into a mold or the like and curing the kneaded product. Accordingly, the deterioration model 1 reproducing the characteristic mechanical strength and fracture characteristics of resin concrete that has deteriorated over time can be produced.

In the kneading process, water may be added to the precursor and then kneaded, but it is desirable to knead the precursor while gradually adding water. Accordingly, water is uniformly dispersed in the whole precursor. Specifically, water is uniformly dispersed between the thermosetting resin and the aggregate (boundary portion). The deterioration model 1 capable of properly reproducing the characteristic mechanical strength and fracture characteristics of the resin concrete can be produced by uniformly dispersing water in the whole precursor. That is, in the deterioration model 1, water exists between the thermosetting resin and the aggregate. Since the structure of such a deterioration model 1 is similar to a structure of resin concrete that has deteriorated over time, the deterioration model 1 can reproduce the characteristic mechanical strength and fracture characteristics of the resin concrete that has deteriorated over time.

In the case of producing a deterioration model 1 simulating resin concrete of an old material age, the addition amount of water (weight of water to be added, hereinafter, it may be referred to as an amount of water added) may be increased. That is, when the deterioration model 1 simulating resin concrete of an old material age is produced, the amount of water added may be increased as compared to the case of producing a deterioration model 1 simulating resin concrete of a young material age. One example of the amount of water added is a weight that is 5% or less of the weight of the precursor (5 parts by weight or less per 100 parts by weight of the precursor, the same applies below). In many cases, a deterioration model of a material age of 50 years or less can be produced with an amount of water added more than 0% and 3% or less.

When the deterioration model 1 is a sample having a predetermined shape (for example, “samples for bending strength, water absorption, and chemical resistance test” in section 7.2 of JIS A 1181: 2005), the kneaded product is injected into a predetermined mold capable of molding into a shape satisfying the standard. For example, a mold capable of molding into a rectangular parallelepiped of 60×60×250 mm may be used as the mold. Then, the kneaded product is cured at room temperature and hardened to such an extent that the kneaded product can be released from the mold. Thereafter, the molded product is taken out of the mold by removing a mold frame. The molded product is cured again at room temperature until hardening completely progresses. Accordingly, the deterioration model 1 having a predetermined shape can be produced.

In the present embodiment, a case in which the deterioration model 1 (refer to FIG. 2) is molded into a rectangular parallelepiped sample having a size of 60×60×250 mm will be exemplified below.

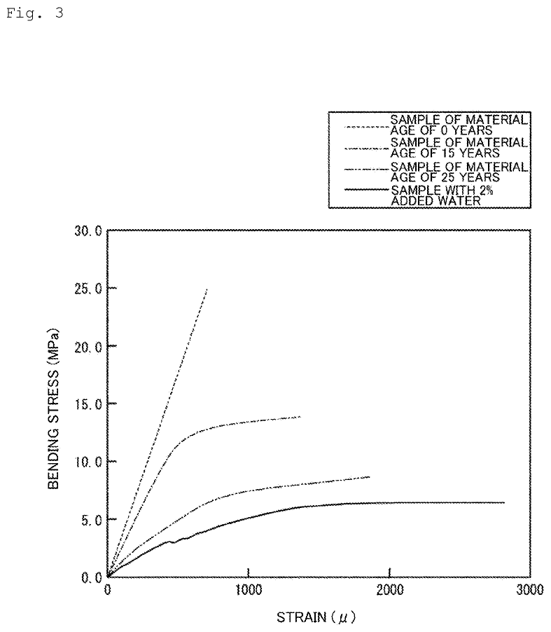

FIG. 3 shows a graph of strain-bending stress of the deterioration model 1 (hereinafter referred to as a sample with 2% added water) with an amount of water added of 2% (2 parts by weight per 100 parts by weight of the precursor) in the formulation shown in Table 1. FIG. 3 shows a graph of a sample with 2% added water superimposed on the graph shown in FIG. 1. The graph of strain-bending stress in the sample with 2% added water is acquired on the basis of “Bending stress test” described in section 8.3 of JIS A 1181:2005, similar to the sample of each material age.

As shown in FIG. 3, the sample with 2% added water shows an elastoplastic fracture behavior, similar to the samples of the material age of 15 years and 25 years and simulates well a state in which resin concrete has deteriorated over time. The bending stress, i.e., the bending strength, at the time of fracture of the sample with 2% added water (deterioration model 1 with an amount of water added of 2%) shown in FIG. 3 is 6.5 MPa. The bending strength of the sample of the material age of 0 years corresponding to a deterioration model 1 with an amount of water added of 0% is 25 MPa.

As described above, in the deterioration model 1, the amount of water added can be increased to simulate resin concrete of an old material age. Therefore, when it is desired to produce a deteriorated model 1 that has deteriorated to a desired strength, the deteriorated model 1 may be produced by adding water corresponding to the strength.

When the deteriorated model 1 deteriorated to a desired strength is to be produced, first, two or more deteriorated models 1 having different amounts of water added are produced as one example. Then, the strengths of the two or more deterioration models 1 having different amounts of water added are acquired (an example of strength evaluation process), and a strength value corresponding to each amount of water added is acquired (an example of data accumulation process). Then, a correlation formula of a value of strength (for example, bending strength) with respect to the amount of water added is acquired (correlation formula acquisition process) on the basis of the acquired two or more strength values and the amounts of water added corresponding to the two or more strength values.

Then, a deterioration model 1 deteriorated to a target strength is produced on the basis of the correlation formula.

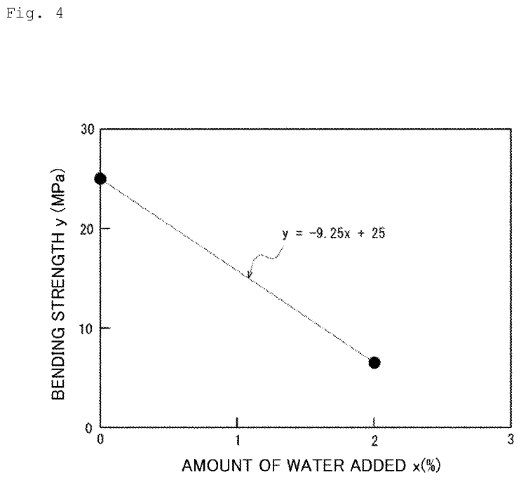

FIG. 4 shows an example of a correlation formula. The correlation formula shown in FIG. 4 is a correlation formula based on a two-point plot of the bending strength (6.5 MPa) and the amount of water added (2%) at the time of fracture of the sample with 2% added water shown in FIG. 3 (deterioration model 1 having an amount of water added of 2%) and a bending strength (25 MPa) and an amount of water added (0%) of a sample of a material age of 0 years corresponding to the deterioration model 1 having an amount of water added of 0%. In FIG. 4, when the amount of water added is x and the bending strength is y, the following formula (1):

$$y = - 9.25 x + 25 \qquad ( 1 )$$

is exemplified as the correlation formula.

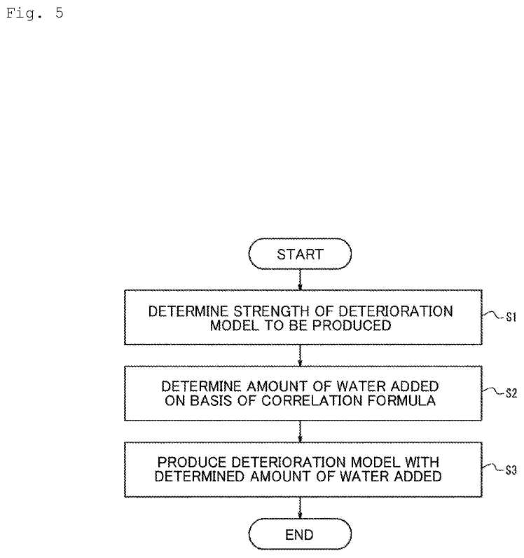

A specific example of producing a deteriorated model 1 that has deteriorated to a target strength on the basis of the correlation formula as described above will be described with reference to the flowchart of FIG. 5. First, a bending strength of a deterioration model 1 to be produced is determined (step S1). For example, the bending strength of the deterioration model 1 to be produced is determined to be 20 MPa.

Next, a target amount of water added (an example of a target addition amount) is determined on the basis of the correlation formula (step S2). Specifically, the bending strength of the deterioration model 1 to be produced determined in the step S1 is inserted into the correlation formula to obtain an amount of water added for producing the deterioration model 1 having the bending strength. For example, when the bending strength of the deterioration model 1 to be produced is 20 MPa, the value of x is about 0.54 when the bending strength is substituted for y in the formula (1). That is, it is possible to predict that the amount of water added for producing the deterioration model 1 having the bending strength of 20 MPa is about 0.54%.

Then, the deterioration model 1 is produced with the amount of water added determined in step S2. For example, the deterioration model 1 having a bending strength of 20 MPa can be produced by producing a deterioration model 1 with an amount of water added of about 0.54%.

The deterioration model 1 produced in this manner is a model simulating resin concrete that has deteriorated to a predetermined extent (of a predetermined material age). That is, various evaluations performed by using the deterioration model 1 are evaluations (an example of a method of predicting deterioration of resin concrete) in which deterioration of resin concrete is predicted. For example, it is possible to realize evaluation of safety of existing structures and design of a structure to be constructed assuming (predicting) strength reduction by using the deterioration model 1 simulating a state deteriorated to a predetermined degree.

In this manner, the method of producing a resin concrete deterioration model, the method of predicting deterioration of resin concrete, and the resin concrete deterioration model can be provided.

### Other Embodiments

(1) The bending strength is exemplified as the strength (mechanical strength) of the deterioration model 1 in the above-described embodiment. However, as the strength of the deterioration model 1, other (mechanical) strengths such as compressive strength and tensile strength may be adopted in addition to the bending strength. In this case, these other strengths may be obtained by measurement or the like as the strength of the deterioration model 1 in the strength evaluation process. The compressive strength and the tensile strength may be acquired on the basis of sections 8.1 to 2 (compressive strength test) or section 8.4 (tensile strength test) of JIS A 1181:2005, similarly to the above-mentioned bending strength.

(2) In the above-described embodiment, a case in which the correlation formula is obtained on the basis of a two-point plot is exemplified. However, acquisition of the correlation formula is not limited to cases in which the correlation formula is acquired on the basis of a two-point plot. For example, the correlation formula may be acquired on the basis of a plot of three points or more. In this case, for example, an approximate formula obtained on the basis of the least squares method or the like may be adopted as a correlation formula. The correlation formula is not limited to a linear formula and may include multidimensional functions of 2 or higher order and exponential functions.

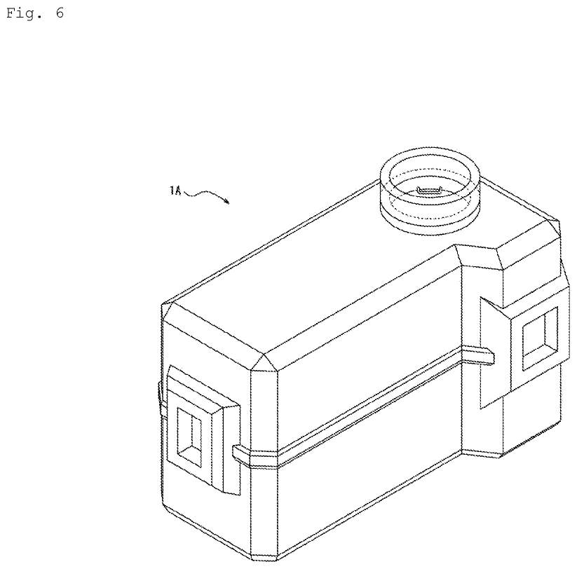

(3) In the above-described embodiment, a case in which the deterioration model 1 (refer to FIG. 2) as a sample is a rectangular parallelepiped having a size of 60×60×250 mm has been exemplified. However, the deterioration model 1 as a sample is not limited to such a shape. For example, as shown in FIG. 6, a resin concrete deterioration model 1A (hereinafter referred to as a deterioration model 1A) simulating the shape of an actual structure may be used. The deterioration model 1A simulating the shape of the actual structure may be used to perform evaluation predicting deterioration of various types of resin concrete. FIG. 6 shows an example of a case in which the deterioration model 1A simulates the shape or structure of a manhole. The deterioration model 1A may simulate a sewer pipe, an information system box, and other structures in addition to a manhole.

The configuration disclosed in the foregoing embodiment (including the other embodiments described above, same as below) can be applied in combination with the configurations disclosed in the other embodiments as long as no contradiction arises. Also, the embodiments disclosed herein are examples, and the embodiments of the invention are not limited thereto and can be modified as appropriate within the scope not departing from the purpose of the present invention.

### INDUSTRIAL APPLICABILITY

The present invention can be applied to a method of producing a resin concrete deterioration model, a method of predicting deterioration of resin concrete, and a resin concrete deterioration model.

### REFERENCE SIGNS LIST

1 Deterioration model (resin concrete deterioration model)
1A Deterioration model (resin concrete deterioration model)

1. A method for producing a resin concrete deterioration model, comprising:
adding water to a precursor of resin concrete containing a thermosetting resin, calcium carbonate, and an aggregate; and
kneading the mixture.

2. The method according to claim 1, further comprising:
mixing the thermosetting resin, the calcium carbonate, and the aggregate to prepare the precursor; and
adding water to the precursor to knead the mixture.

3. The method according to claim 1, wherein an addition amount of the water is increased when a deterioration model simulating a case of an older material age is produced.

4. A method of predicting deterioration of resin concrete, comprising:
a model producing step of producing a resin concrete deterioration model, the resin concrete deterioration model containing a thermosetting resin, calcium carbonate, and aggregate;
a strength evaluation step of acquiring a strength of the resin concrete deterioration model;
a data accumulation step of producing two or more resin concrete deterioration models having different addition amounts of water in the model producing step;
executing the strength evaluation step to acquire a value of the strength corresponding to the addition amount of water; and
a correlation formula acquisition step of acquiring a correlation formula of the strength with respect to the addition amount on the basis of the acquired two or more strengths and the addition amount corresponding to the two or more strengths.

5. The method according to claim 4, wherein the resin concrete deterioration model deteriorated to a target strength is produced on the basis of the correlation formula.

6. The method according to claim 5,
wherein a target addition amount of the water corresponding to the target strength is obtained on the basis of the correlation formula, and
the target addition amount of the water is added to a precursor of resin concrete containing the thermosetting resin, the calcium carbonate, and the aggregate to produce the resin concrete deterioration model.

7. The method according to claim 5, wherein the strength is a bending strength.

8. The method according to claim 4, wherein the resin concrete deterioration model comprising:
a thermosetting resin,
calcium carbonate,
an aggregate, and
water.

9. The method according to claim 1, wherein the resin concrete deterioration model simulates a mechanical strength of resin concrete of a predetermined material age.

10. The method according to claim 1, wherein the adding water further comprises gradually adding water to the mixture while kneading the mixture.

11. The method according to claim 1, wherein the aggregate includes at least one of sand or gravel.

12. The method according to claim 1, wherein the thermoset resin includes at least one of an unsaturated polyester resin, a phenol resin, or an epoxy resin.

13. The method according to claim 1, wherein adding more water produces the resin concrete deterioration model that simulates an older material age.

14. The method according to claim 2, wherein an addition amount of the water is increased when a deterioration model simulating a case of an older material age is produced.

15. The method according to claim 4, wherein the resin concrete deterioration model simulates a mechanical strength of resin concrete of a predetermined material age.

16. The method according to claim 4, wherein the model producing step further comprises gradually adding water to the resin concrete deterioration model while kneading the resin concrete deterioration model.

17. The method according to claim 4, wherein the aggregate includes at least one of sand or gravel.

18. The method according to claim 4, wherein the thermoset resin includes at least one of an unsaturated polyester resin, a phenol resin, or an epoxy resin.
